SEQUENCE LISTING (1 sequence)

SEQ ID NO: 1 - protein, 4 aa, Artificial Sequence
  Description of Artificial Sequence Synthetic polypeptide
WPRE

(19) United States
(12) Patent Application Publication
(10) Pub. No.: US 2023/0323302 A1
(43) Pub. Date: Oct. 12, 2023

(54) COMBATING COVID-19 USING ENGINEERED INKT CELLS

(71) Applicant: The Regents of the University of California, Oakland, CA (US)

(72) Inventors: Lili Yang, Los Angeles, CA (US); Yan-Ruide Li, Los Angeles, CA (US)

(73) Assignee: The Regents of the University of California, Oakland, CA (US)

(21) Appl. No.: 18/044,000

(22) PCT Filed: Sep. 3, 2021
(86) PCT No.: PCT/US21/49083

§ 371 Date: Mar. 3, 2023

Related U.S. Application Data
(60) Provisional application No. 63/076,494, filed on Sep. 10, 2020.

(51) Int. Cl. C12N 5/0783; C07K 14/725

(52) U.S. Cl. CPC C12N 5/0646; C07K 14/7051

(57) ABSTRACT

Aspects of the present disclosure relate to methods and compositions related to the preparation of immune cells, including engineered invariant natural killer T (iNKT) cells for off-the-shelf use for COVID-19 clinical therapy. The iNKT cells may be produced from hematopoietic stem progenitor cells and are suitable for allogeneic cellular therapy.

### CROSS-REFERENCE TO RELATED APPLICATIONS

This application claims the benefit under 35 U.S.C. Section 119(e) of co-pending and commonly-assigned U.S. Provisional Patent Application Ser. No. 63/076,494, filed on Sep. 10, 2020, and entitled “COMBATING COVID-19 USING ENGINEERED INKT CELLS” which application is incorporated by reference herein.

### TECHNICAL FIELD

Embodiments of the disclosure concern at least the fields of immunology, cell biology, molecular biology, and medicine.

### BACKGROUND OF THE INVENTION

Severe acute respiratory syndrome coronavirus 2 (SARS-CoV-2), closely related to SARS-CoV, is an enveloped, single-stranded positive RNA virus with a nucleocapsid that belongs to the betacoronavirus genus of the Coronaviridae. The novel SARS-CoV-2 is the cause of the coronavirus disease 19 (COVID-19) pandemic. Patients who are over 60 years of age with underlying conditions are at high risk for severe COVID-19, which is associated with a 75% risk for mechanical ventilation and 50% risk of death.

The virus is primarily spread between people during close contact, (a) most often via small droplets produced by coughing, (b) sneezing, and talking. The droplets usually fall to the ground or onto surfaces rather than travelling through air over long distances. The time from exposure to onset of symptoms is typically around five days but may range from two to fourteen days. Common symptoms include fever, cough, fatigue, shortness of breath, and loss of smell and taste. While the majority of cases result in mild symptoms, some progress to acute respiratory distress syndrome (ARDS), multi-organ failure, septic shock, and blood clots. With the uprising new cases worldwide, there are increasing concerns that COVID-19 may stay/recur within the human society for an extended period, and that a vaccine may not be adequate to end COVID-19.

There is a need for therapeutic agents and associated methods designed to help individuals suffering from COVID-19 infection. The present disclosure provides solutions to an urgent need for COVID-19 therapies.

### SUMMARY OF THE INVENTION

In the absence of an effective treatment for COVID-19, this accelerating threat to human health must be met with speedy development of innovative therapeutics. As discussed in detail below, cell therapy represents a very promising approach for COVID-19 therapies. In particular, invariant natural killer T (iNKT) cells are powerful innate immune cells that have potential to clear virus infection and mitigate harmful inflammation. Meanwhile these cells have demonstrated strong safety profile in the oncology clinic and confer no graft-versus-host (GvHD).

The invention disclosed herein provides new methods and materials for making and using hematopoietic stem cell (HSC)-engineered immune cells, and engineered natural killer T (iNKT) cells in particular that are useful, for example, in methods of treating an individual suffering from coronavirus disease 19. HSC-engineered immune cell embodiments of the invention also include compositions of matter comprising an engineered HSC cell in combination with a severe acute respiratory syndrome coronavirus 2 (SARS-CoV-2), for example one disposed within an infected cell, or presented on an antigen presenting cell.

Illustrative methods of the invention include disposing an engineered natural killer T (iNKT) cell disclosed herein in an individual suffering from coronavirus disease 19 such that the iNKT cells kill host cells in the individual that are infected with SARS-CoV-2. In typical embodiments of the invention, the engineered iNKT cell comprises one or more exogenous nucleic acids such as those encoding a T cell receptor alpha chain, a T cell receptor beta chain, a suicide gene or the like. In certain embodiments, the genome of the engineered iNKT cell has been altered to eliminate surface expression of at least one HLA-I and/or HLA-II molecule.

Illustrative embodiments of the invention include methods of preparing an engineered natural killer T (iNKT) cell, the methods typically comprising selecting stem or progenitor cells; introducing one or more nucleic acids encoding, for example nucleic acids encoding a suicide gene, or nucleic acids encoding at least one T-cell receptor (TCR) alpha and/or beta chain gene into the stem or progenitor cells; and then culturing the cells to induce the differentiation of the cells into invariant natural killer T (iNKT) cells so that a natural killer T (iNKT) cell is prepared. Typically, in these methods, the engineered natural killer T (iNKT) cell comprises an exogenous suicide gene; and/or the genome of the cell has been altered to eliminate surface expression of at least one HLA-I or HLA-II molecule. In certain embodiments of the invention, at least one TCR is expressed from an exogenous nucleic acid and/or from an endogenous invariant TCR gene that is under the transcriptional control of a recombinantly modified promoter region.

Embodiments of the invention also include compositions of matter comprising an engineered invariant natural killer T (iNKT) cell, for example one that comprises a gene expression profile characterized as being HLA-I-negative; HLA-II-negative; HLA-E-positive; and/or expressing a suicide gene. In certain embodiments of the invention the composition further includes a severe acute respiratory syndrome coronavirus 2 (SARS-CoV-2), for example one disposed within an infected cell. In typical embodiments of the invention, one or more exogenous nucleic acids are transduced into stem or progenitor cells to make the engineered iNKT cell. In certain embodiments of the invention, the engineered iNKT cell is disposed in an allogeneic in vivo environment (e.g. in embodiments of the invention that include methods of treating an individual infected with SARS-CoV-2 by administering an engineered iNKT cell as disclosed herein).

In illustrative embodiments of the invention, CB CD34⁺ HSCs can be obtained from commercial providers (e.g., HemaCare) or from established CB banks. Cells can be shipped to central GMP-facilities for manufacturing, testing, formulation, and cryopreservation. Once passed the lot release testing, the cell product can be shipped to hospitals for direct infusion. Because the HSC-iNKT cellular product is an off-the-shelf product that can be used to treat COVID-19 patients independent of MHC restrictions, once commercialized, this cellular product can allow a broad application of this potentially life-saving stem cell-based therapy.

Autologous gene-modified cellular therapy, like the newly approved Kymriah and Yescarta (CAR-T therapy), has a market price of ˜$300-500k per patient per treatment. An off-the-shelf product, like the allogeneic HSC-iNKT cells disclosed herein, can greatly reduce cost. The cost of manufacturing one batch of HSC-iNKT cells may be higher than that of manufacturing CAR-T cells, but unlikely will differ by over 10-fold. Even assuming a 10-fold higher manufacturing cost, the proposed HSC-iNKT cell therapy will still only cost ˜$3-5k per dose (per patient per treatment), making the therapy reasonably affordable.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1E. In Vitro Generation and Characterization of Allogenic HSC-Engineered iNKT (AlloHSC-iNKT) Cells. (a) Experimental design to generate AlloHSC-iNKT cells in vitro. CB, cord blood; HSC, hematopoietic stem cell; SG, suicide gene; Lenti/iNKT-SG, lentiviral vector encoding an iNKT TCR gene and a suicide gene; ATO, artificial thymic organoid. (b) Generation of iNKT cells (identified as iNKT TCR+CD3+ cells) during ATO culture. A 6B11 monoclonal antibody was used to stain iNKT TCR. (c) Generation of iNKT cells (identified as iNKT TCR+CD3+ cells) during Feeder-free culture. (d) Yields of AlloHSC-iNKT cells generated from ATO and Feeder-free cultures. (e) FACS characterization of surface marker expression and intracellular cytokine and cytotoxic molecule production of AlloHSC-iNKT cells. Periphery blood mononuclear cell (PBMC)-derived iNKT (PBMC-iNKT) cells and conventional αβ T (PBMC-Tc) cells were included as controls. Representative of over experiments.

FIGS. 2A-2I. AlloHSC-iNKT Cells Directly Target and Kill SARS-CoV-2 Infected Cells.

(a) Schematics showing the engineered 293T-FG, 293T-ACE2-FG, and Calu3-FG cell lines. ACE2, angiotensin converting enzyme 2; Fluc, firefly luciferase; EGFP, enhanced green fluorescent protein; F2A, foot-and-mouth disease virus 2A self-cleavage sequence. (b) FACS detection of ACE2 on 293T-FG, 293T-ACE2-FG, Calu3-FG, and AlloHSC-iNKT cells. (c-h) In vitro direct killing of SARS-CoV-2 infected cells by ATO culture-generated AlloHSC-iNKT cells. (c) Experimental design. (d) Target cell killing data of AlloHSC-iNKT cells at 24-hours post co-culturing with infected cells (n=5). (e) FACS detection of CD69, Perforin and Granzyme B of AlloHSC-iNKT cells at 24-hours post co-culturing with SARS-CoV-2 infected 293T-ACE2-FG cells. (0 ELISA analysis of IFN-γ production (n=3). (h) SARS-CoV-2 infected cell killing mechanisms of AlloHSC-iNKT cells. NKG2D and DNAM-1 mediated pathways were studied (n=5). (i) Immunofluorescence analysis of direct targeting of SARS-CoV-2 infected cells by AlloHSC-iNKT cells. Representative of 3 experiments. Data are presented as the mean±SEM. ns, not significant, *P<0.05, **P <0.01, ***P<0.001, ****P<0.0001, by Student's t test (d, f and g), or by 1-way ANOVA (h). See also Figure Sl.

FIGS. 3A-3E. AlloHSC-iNKT Cells Reduce Virus-Infection Promoted Inflammatory Monocytes. (a) Experimental design. 293T-ACE2-FG cells were infected by SARS-CoV-2 virus. After 1 day, ATO culture-generated AlloHSC-iNKT cells and donor-mismatched PBMCs were added and incubated for 24 hours. Flow cytometry was used to detect cell populations. (b) FACS detection of CD14+ monocytes, T cells, and B cells in PBMCs. (c) Quantification of (b) (n=5). (d) FACS detection of CD1d expression on CD14+ monocytes, T cells, and B cells. (e) Quantification of (d) (n=5). Representative of 3 experiments. Data are presented as the mean±SEM. ns, not significant, *P<0.05, **P<0.01, ***P<0.001, ****P<0.0001, by 1-way ANOVA.

FIGS. 4A-4J. Safety and Immunogenicity of AlloHSC-iNKT Cells. (a-b) Studying the graft-versus-host (GvH) response of AlloHSC-iNKT cells using an in vitro mixed lymphocyte reaction (MLR) assay. PBMC-Tc cells were included as a responder cell control. (a) Experimental design. PBMCs from 5 different healthy donors were used as stimulator cells. (b) ELISA analyses of IFN-γ production at day 4 (n=3). N, no stimulator cells. (c-d) Studying the GvH response of AlloHSC-iNKT cells using NSG mouse model. PBMC-Tc were included as a control. (A) Experimental design. (B) Kaplan-Meier survival curves of experimental mice over time (n=6). (e-g) Studying T cell-mediated alloreaction against AlloHSC-iNKT cells using an in vitro MLR assay. Irradiated AlloHSC-iNKT cells (as stimulators) were co-cultured with donor-mismatched PBMC cells (as responders). Irradiated PBMC-Tc cells were included as a stimulator cell control. (e) Experimental design. PBMCs from 3 different healthy donors were used as responders. (0 ELISA analyses of IFN-γ production at day 4 (n=3). (g) FACS analyses of HLA-I and HLA-II expression on the indicated stimulator cells (n=5). (h j) Studying HLA-I and HLA-II expression on AlloHSC-iNKT cells under SARS-CoV-2 infection. AlloHSC-iNKT cells were co-cultured with SARS-CoV-2 infected target cells. PBMC-Tc cells were included as a control. (h) Experimental design. (i) FACS analyses of HLA-I and HLA-II expression on the indicated stimulator cells. (j) Quantification of (i) (n=5). Representative of 3 experiments. Data are presented as the mean±SEM. ns, not significant, *P<0.05, **P <0.01, ***P<0.001, ****P<0.0001, by Student's t test (j and g), by 1-way ANOVA (b and f), or by log rank (Mantel-Cox) test adjusted for multiple comparisons (d).

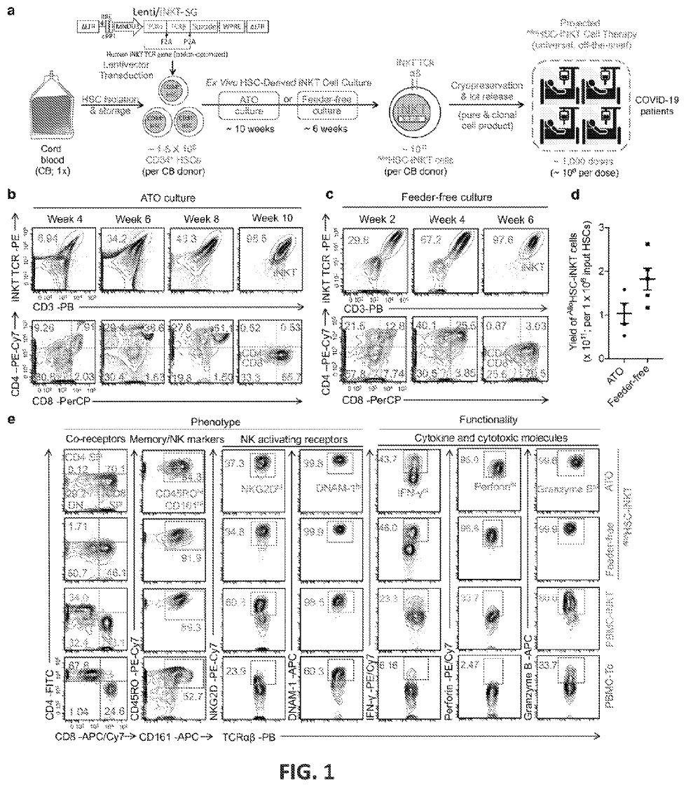

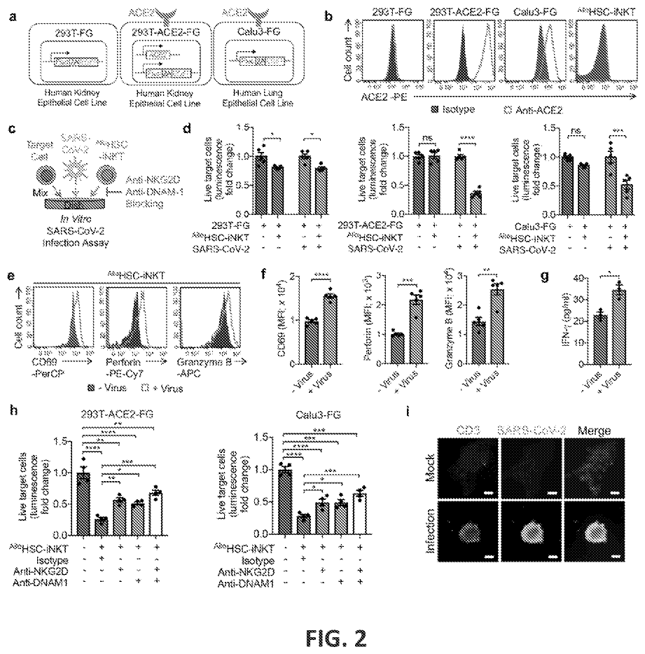

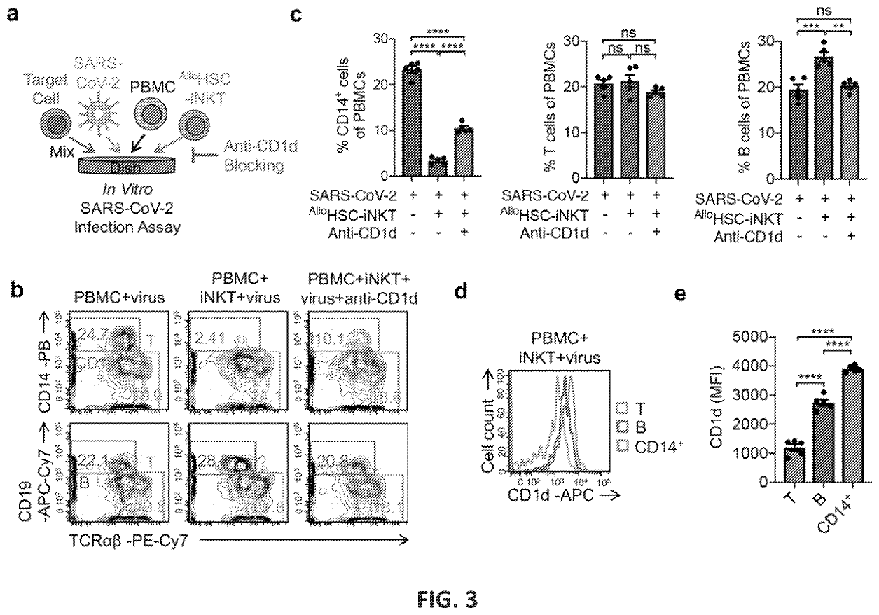

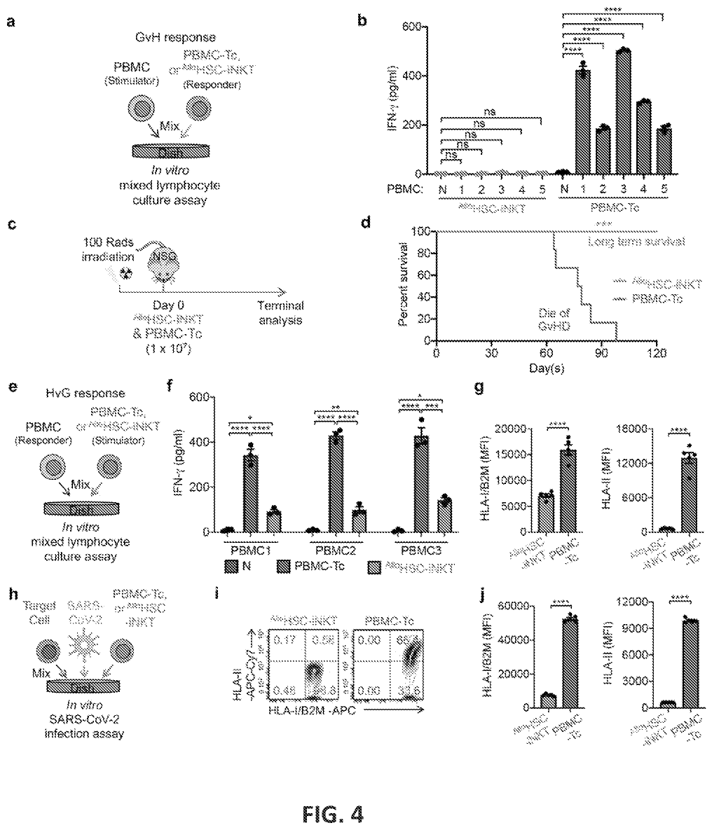

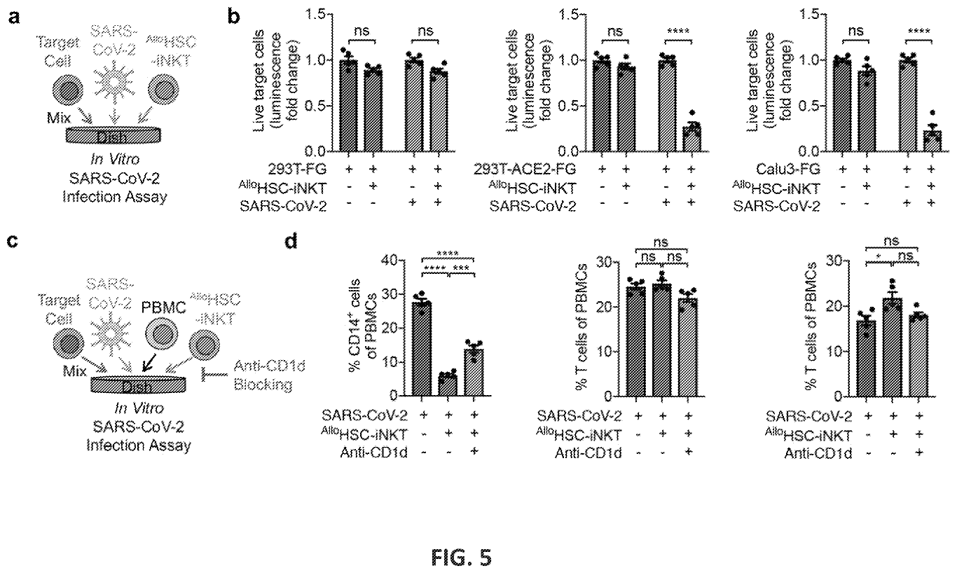

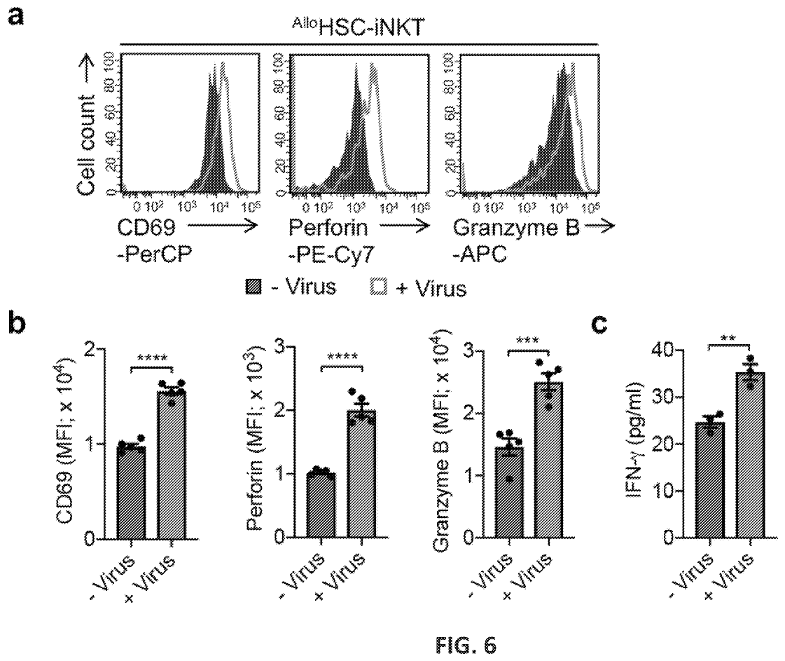

FIGS. 5A-5D. Reduction of SARS-CoV-2 Virus Infection Load and Virus Infection-Induced Hyperinflammation by Feeder-Free Culture-Generated AlloHSC-iNKT Cells; Related to FIGS. 1 and 2. (a-b) Study directly targeting of SARS-CoV-2 infected cells by Feeder-free culture-generated AlloHSC-iNKT cells. (a) Experimental design. (b) Target cell killing data of AlloHSC-iNKT cells at 24-hours post co-culturing with infected cells (n=5). (c-d) Study targeting virus-infection promoted inflammatory monocytes by Feeder-free culture-generated AlloHSC-iNKT cells. (c) Experimental design. (d) Flow cytometry analysis of remaining CD14+ monocytes, T and B cells in PBMCs after co-culturing with AlloHSC-iNKT cells. Representative of 3 experiments. Data are presented as the mean±SEM. ns, not significant, *P<0.05, **P<0.01, ***P<0.001, ****P<0.0001, by Student's t test (b) and 1-way ANOVA (d).

FIGS. 6A-6C. AlloHSC-iNKT Cells are Activated by SARS-CoV-2 Infected Target Cells; Related to FIG. 1. (a) FACS detection of CD69, Perforin, and Granzyme B of AlloHSC-iNKT cells at 24-hours post co-culturing with SARS-CoV-2 infected Calu3-FG cells. (b) Quantification of (a) (n=5) (c) ELISA analysis of IFN-γ production (n=3). Representative of 3 experiments. Data are presented as the mean±SEM. ns, not significant, *P<0.05, **P<0.01, ***P<0.001, ****P<0.0001, by Student's t test.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

New COVID-19 treatments are desperately needed as case numbers continue to rise and emergent strains threaten vaccine efficacy. Cell therapy has revolutionized cancer treatment and holds much promise in combatting infectious disease, including COVID-19. Invariant natural killer T (iNKT) cells are a rare subset of T cells with potent antiviral and immunoregulatory functions and an excellent safety profile. Current iNKT cell strategies are hindered by the extremely low presence of iNKT cells, and we have developed a platform to overcome this critical limitation.

As discussed in detail below, we produced allogeneic HSC-engineered iNKT (^{Allo}HSC-iNKT) cells through TCR engineering of human cord blood CD34⁺ hematopoietic stem cells (HSCs) and differentiation of these HSCs into iNKT cells in an Ex Vivo HSC-Derived iNKT Cell Culture. We then established in vitro SARS-CoV-2 infection assays to assess ^{Allo}HSC-iNKT cell antiviral and anti-hyperinflammation functions. Lastly, using in vitro and in vivo preclinical models, we evaluated ^{Allo}HSC-iNKT cell safety and immunogenicity for off-the-shelf application.

We reliably generated ^{Allo}HSC-iNKT cells at high-yield and of high-purity; these resulting cells closely resembled endogenous human iNKT cells in phenotypes and functionalities. In cell culture, ^{Allo}HSC-iNKT cells directly killed SARS-CoV-2 infected cells and also selectively eliminated SARS-CoV-2 infection-stimulated inflammatory monocytes. In an in vitro mixed lymphocyte reaction (MLR) assay and an NSG mouse xenograft model, ^{Allo}HSC-iNKT cells were resistant to T cell-mediated alloreaction and did not cause GvHD.

As discussed in detail below, the invention disclosed herein has a number of embodiments. Embodiments of the invention include methods of inhibiting replication of severe acute respiratory syndrome coronavirus 2, for example methods useful in therapeutic regimens designed to treat individuals suffering from coronavirus disease 19. In view of the manner in which iNKT cells target infected cells, the variety of variants of the severe acute respiratory syndrome coronavirus 2 (SARS-CoV-2), can be targeted using the methods disclosed herein. Such methods include combining invariant natural killer T (iNKT) cells with cells infected with one or more variants of the severe acute respiratory syndrome coronavirus 2 (e.g., alpha, beta, gamma, delta or other variant) in methods designed to inhibit replication of such severe acute respiratory syndrome coronavirus 2 (i.e., including variants).

Typically these methods include combining a plurality of purified invariant natural killer T cells with cells infected with the severe acute respiratory syndrome coronavirus 2 (e.g. human monocyte cells) such that the invariant natural killer T cells selectively kill the cells infected with the severe acute respiratory syndrome coronavirus 2, thereby inhibiting replication of severe acute respiratory syndrome coronavirus 2. Typically in these methods the plurality of purified invariant natural killer T cells comprise allogeneic CD34+ hematopoietic stem cell derived engineered invariant natural killer T cells. Using such methods, a plurality of purified invariant natural killer T cells can be combined in vivo with cells infected with the severe acute respiratory syndrome coronavirus 2 so as to treat an individual suffering from coronavirus disease 19. In certain methods of the invention, the plurality of purified invariant natural killer T cells comprises at least 1×10⁶ or 1×10⁷ or 1×10⁸ invariant natural killer T cells. Optionally, the method comprises using a plurality of purified invariant natural killer T cells that are thawed following cryopreservation.

There are a number of embodiments of the methods disclosed herein. For example, in certain embodiments of the invention, the engineered iNKT cells used in the methods are obtained by transducing one or more exogenous nucleic acids into CD34⁺ stem or progenitor cells such that the engineered iNKT cells are made (see, e.g., PCT Application Serial No. PCT/US19/36786, the contents of which are incorporated by reference). In certain methods of the invention, the one or more exogenous nucleic acids transduced into the stem or progenitor cells comprise a T cell receptor alpha chain gene and/or a T cell receptor alpha chain gene; and/or the engineered iNKT cells comprise clonal populations of cells comprising the T cell receptor alpha chain gene and/or the T cell receptor beta chain gene. In typical embodiments of the invention, the iNKT cells express a canonical invariant TCR α chain (e.g. Vα24-Jα18 in humans) that is typically complexed with a semi-variant TCR β chain (e.g. Vβ11 in humans), a TCR that recognizes lipid antigens presented by CD1d, a non-polymorphic MHC Class I-like protein. Optionally in these methods, the engineered iNKT cell comprises one or more exogenous nucleic acids encoding at least one functional T-cell receptor (TCR), wherein the TCR recognizes one or more SARS-CoV-2 antigens. In some methods of the invention, the plurality of purified invariant natural killer T cells comprise engineered invariant natural killer T cells further comprise a gene expression profile characterized as: HLA-I-negative; and/or HLA-II-negative; and/or HLA-E-positive; and/or expressing a suicide gene.

Other embodiments of the invention include compositions of matter comprising a plurality of purified invariant natural killer T cells; and cells infected with the severe acute respiratory syndrome coronavirus 2 (e.g. human monocyte cells). In such compositions, the plurality of purified invariant natural killer T cells can comprise allogeneic CD34⁺ hematopoietic stem cell derived engineered invariant natural killer T cells. Typically in these compositions, the engineered iNKT cells comprise one or more exogenous nucleic acids transduced therein. For example, in certain embodiments of the invention, the one or more exogenous nucleic acids comprise a T cell receptor alpha chain gene and/or a T cell receptor alpha chain gene; and the engineered iNKT cells comprise clonal populations of cells comprising the T cell receptor alpha chain gene and/or the T cell receptor beta chain gene. In some embodiments of the invention, the engineered iNKT cell comprises one or more exogenous nucleic acids encoding at least one functional T-cell receptor (TCR), wherein the TCR recognizes one or more SARS-CoV-2 antigens. In certain compositions of the invention, the plurality of purified invariant natural killer T cells comprise engineered invariant natural killer T cells further comprise a gene expression profile characterized as HLA-I-negative; and/or HLA-II-negative; and/or HLA-E-positive; and/or expressing a suicide gene. Optionally, the plurality of purified invariant natural killer T cells in the composition comprises at least 1×10⁶ or 1×10⁷ or 1×10⁸ invariant natural killer T cells.

In certain embodiments of the invention, the composition includes a pharmaceutically acceptable excipient selected from at least one of a preservative, a tonicity adjusting agent, a detergent, a hydrogel, a viscosity adjusting agent, or a pH adjusting agent. Such pharmaceutically acceptable excipients are well known in that art and a thorough discussion of pharmaceutically acceptable carriers, diluents, and other excipients is presented in Remington's Pharmaceutical Sciences (Mack Pub. Co., N.J. current edition). Further aspects and embodiments of the invention are discussed in the following sections.

As discussed in detail below, we report a method to robustly produce therapeutic levels of ^{Allo}HSC-iNKT cells. Preclinical studies showed that these ^{Allo}HSC-iNKT cells closely resembled endogenous human iNKT cells, could reduce SARS-CoV-2 virus infection load and mitigate virus infection-induced hyperinflammation, and meanwhile were free of GvHD-risk and resistant to T cell-mediated allorejection. These results support the development of ^{Allo}HSC-iNKT cells as a promising off-the-shelf cell product for treating COVID-19; such a cell product has the potential to target the new emerging SARS-CoV-2 variants as well as the future new emerging viruses.

SARS-CoV-2, the etiologic agent of the COVID-19 pandemic, is responsible for over 30 million cases and 600 thousand deaths in the United States alone (1). As case numbers continue to rise, there are increasing concerns that COVID-19 may stay/recur within human society for an extended period (2), and that vaccines, although highly effective and produced with unprecedented speed (3-5), may not be adequate to end COVID-19 (6). Positive cases in vaccine recipients can occur (7), emergent strains may evade memory responses (8), and for several reasons significant proportions of society are unvaccinated (9). Despite tremendous efforts to generate antiviral drugs and therapeutic interventions, including nucleoside analogs (10, 11), chloroquine (10), protease inhibitors (12), monoclonal antibody therapy (13-15), and cell-based therapy (16, 17), only one drug, remdesivir, has received FDA approval for treating COVID-19, notwithstanding an absence of survival benefit (18). To reduce COVID-19 mortality, novel therapies are urgently needed.

Cell-based immunotherapy has reshaped cancer treatment (19-21) and shown strong clinical efficacy in the treatment of infectious disease (22-24), and is now being investigated for COVID-19 (16, 17). A recent study of critically ill COVID-19 patients reported the functional alteration of innate T cells, including invariant natural killer T (iNKT) and mucosal associated invariant T (MAIT) cells, showing that the patients contained significantly reduced numbers of iNKT cells and the activation status of iNKT cells was predictive of disease severity, suggesting the involvement of iNKT cells in COVID-19 (25). iNKT cells are a unique subpopulation of T cells expressing a canonical invariant TCR α chain (Vα24-Jα18 in human) complexed with a semi-variant TCR β chain (mainly Vβ11 in human) that recognizes lipid antigens presented by CD1d, a non-polymorphic MHC Class I-like protein (26). These cells play an important role in linking innate and adaptive immune responses and have been implicated in infectious disease, allergy, asthma, autoimmunity, and tumor surveillance (26-28). A growing body of work indicates that iNKT cells play a beneficial role in battling acute respiratory virus infection, as these cells were shown to boost early innate immune responses, reduce viral titer, and inhibit the suppressive capacity of myeloid-derived suppressor cells (MDSCs) to enhance virus-specific responses in influenza models (28-32). iNKT cells also reduced the accumulation of inflammatory monocytes in the lungs and decreased immunopathology during severe influenza A virus infection (29), demonstrating the potential for iNKT cells to have dual antiviral functions by direct virus clearance and inflammatory monocyte regulation. Importantly, because iNKT cells do not recognize mismatched MHC molecules and protein autoantigens, they are not expected to cause graft-versus-host disease (GvHD) and therefore are suitable for developing allogeneic “off-the-shelf” cell therapy targeting COVID-19 (33-35).

Current iNKT cell-based therapies are restricted by the extremely low number and high variability of iNKT cells in peripheral blood (36, 37). To overcome this critical limitation, we genetically engineered hematopoietic stem cells (HSCs) to express the iNKT TCR, which engendered the in vivo lineage commitment and expansion of both mouse and human HSC engineered iNKT (HSC-iNKT) cells following bone marrow transfer (38, 39). However, such an in vivo approach can only be translated for autologous HSC adoptive therapies (39). Recently, we have developed an Ex Vivo HSC-Derived iNKT Cell Culture method that can robustly produce therapeutic levels of allogeneic HSC-engineered human iNKT (^{Allo}HSC-iNKT) cells. Here, we report the preclinical study of an ^{Allo}HSC-iNKT cell therapy, showing its feasibility, safety, and COVID-19 therapy potential.

On Jun. 2, 2020, FDA approved an allogeneic iNKT cell therapy for treating COVID-19 patients. However, most allogeneic iNKT cell products are expanded from peripheral blood of healthy donors. Such approaches have several critical limitations, including the extremely low number and high variability of iNKT cells in blood to start with (˜0.001-1%). Additional problems include the possible presence of bystander conventional αβ T cells that risk inducing GvHD. To overcome these critical limitations, we have developed a strategy to produce therapeutic levels of pure and clonal allogeneic hematopoietic stem cell-engineered iNKT (HS C-iNKT) cells, initially for targeting cancer. As disclosed below, we can utilize allogeneic HSC-iNKT cell therapy for targeting COVID-19. From a single cord blood (CB) donor, about 10¹¹ allogeneic HSC-iNKT cells can be generated that can be formulated into 1,000 doses and cryopreserved for storage and readily distribution to treat COVID-19 patients, thereby addressing an important and unmet medical need.

Generation of Allogeneic HSC-Engineered iNKT (^{Allo}HSC-iNKT) Cells

Human cord blood (CB) CD34⁺ hematopoietic stem and progenitor cells (denoted as HSCs) were transduced with a Lenti/iNKT-SG vector and then differentiated into iNKT cells in an Ex Vivo HSC-Derived iNKT (HSC-iNKT) cell culture, using either an Artificial Thymic Organoid (ATO) culture system or a Feeder-Free culture system (FIG. 1a). The Lenti/iNKT-SG vector has been previously used to generate autologous HSC-engineered iNKT cells for cancer immunotherapy (39); Depending on the needs, in the same lentivectors we can include a suicide gene (SG) (e.g., sr39TK) to provide cell products with a “safety switch” (39); ATO is 3D cell culture system supporting the ex vivo differentiation of human T cells from HSCs (40, 41); the Feeder-Free culture adopts a system of plate-bound delta-like 4 (DLL4) and vascular cell adhesion protein 1 (VCAM-1) to enable T lymphoid differentiation (42-45). Lentivector transduced HSCs were seeded in ATO culture or Feeder-free culture, where HSCs differentiated into human iNKT cells over a course of 10 weeks or 6 weeks, respectively, resulting in pure and clonal ^{Allo}HSC-iNKT cells without bystander conventional αβ T cells (FIG. 1a-1c). During the Ex Vivo HSC-derived iNKT cell cultures, ^{Allo}HSC-iNKT cells followed a typical iNKT cell development path defined by CD4/CD8 co-receptor expression (36). ^{Allo}HSC-iNKT cells transitioned from CD4⁻CD8⁻ to CD4⁺CD8⁺, then to CD4⁻CD8^{+/-} (FIGS. 1b and 1c). At the end of cultures, most of the ^{Allo}HSC-iNKT cells demonstrated a CD4⁻CD8^{+/-} phenotype (FIGS. 1b and 1c).

The manufacturing process of generating ^{Allo}HSC-iNKT cells using either ATO culture or Feeder-free culture were robust and of high yield and high purity for all CB donor tested (FIG. 1d). Based on the results, it was estimated that from one single CB donor (comprising ˜1-5×10⁶ HSCs), ˜10^{11 Allo}HSC-iNKT cells could be generated that can potentially be formulated into ˜1,000 doses (FIG. 1d) (46-49). The ^{Allo}HSC-iNKT cell products contained pure transgenic iNKT cells and nearly undetectable bystander conventional αβ T cells, therefore reducing GvHD risk and supporting the use of ^{Allo}HSC-iNKT cells as an off-the-shelf therapy.

### Phenotype and Functionality of ^{Allo}HSC-iNKT Cells

To study their phenotype and functionality, we compared ^{Allo}HSC-iNKT cells to healthy donor periphery blood mononuclear cell (PBMC)-derived iNKTs and conventional αβ T cells (denoted as PBMC-iNKT and PBMC-Tc cells, respectively). Both ATO and Feeder-Free cultured ^{Allo}HSC-iNKT cells displayed typical iNKT cell phenotype similar to that of PBMC-iNKT cells, but distinct from that of PBMC-Tc cells. ^{Allo}HSC-iNKT cells presented as CD4⁻CD8^{+/-} cells and expressed high levels of memory T cell marker CD45RO and NK cell marker CD161 (FIG. 1e). In addition, compared to PBMC-iNKT and PBMC-Tc cells, ^{Allo}HSC-iNKT cells upregulated NK activating receptors like NKG2D and DNAM-1 and produced exceedingly high levels of the effector cytokine IFN-γ and cytotoxic molecules like Perforin and Granzyme B (FIG. 1e), indicating the potent effector potential of ^{Allo}HSC-iNKT cells. Despite the manufacturing difference, ^{Allo}HSC-iNKT cells generated from ATO culture or Feeder-free culture displayed similar phenotype and functionality (FIG. 1e); in this report, these cells were alternatively used and showed comparable COVID-19 targeting potential.

### Direct Killing of SARS-CoV-2-Infected Cells by ^{Allo}HSC-iNKT Cells

Following the successful generation of ^{Allo}HSC-iNKT cells, we established an in vitro SARS-CoV-2 infection assay to assess the direct killing of SARS-CoV-2-infected cells. Studies have indicated that SARS-CoV-2 can infect multiple tissues in addition to lung tissue (50, 51). We therefore established in vitro infection models using two cell lines: a human kidney epithelial cell line, 293T, and a human lung epithelial cell line, Calu-3 (FIGS. 2a and 2b). The parental 293T cell line does not express ACE2, and we engineered a subline to overexpress ACE2 (FIG. 2b). All target cell lines were also engineered to express a firefly luciferase (Fluc) and green fluorescence protein (EGFP) dual-reporters (FIG. 2b). As a result, three target cell lines were generated, 293T-FG, 293T-ACE2-FG, and Calu3-FG, with 293T-FG serving as a negative control for studying SARS-CoV-2 infection (FIGS. 2a and 2b). Notably, ^{Allo}HSC-iNKT cells do not express ACE2, indicating that these therapeutic cells themselves are not susceptible to SARS-CoV-2 infection (FIG. 2b). ^{Allo}HSC-iNKT cells effectively and selectively killed 293T-ACE2-FG and Calu3-FG cells, but not the 293T-FG control cells, under SARS-CoV-2 infection conditions. This suggests that ^{Allo}HSC-iNKT cells can specifically target SARS-CoV-2 infected cells without damaging uninfected tissue (FIGS. 2c, 2d, 5a, and 5b). The killing of virus-infected target cells was associated with the activation of ^{Allo}HSC-iNKT cells, as shown by their upregulated expression of activation markers (i.e., CD69) and production of effector molecules (i.e., Perforin, Granzyme B, and IFN-γ) (FIGS. 2e-2g, 6a and 6b). In addition, the target cell killing was significantly reduced by blocking NKG2D and DNAM-1, indicating an NK activation receptor-mediated effector mechanism (FIG. 2h). Corroborating the cytotoxicity towards virus-infected cells, immunohistology imaging studies showed the selective clustering of ^{Allo}HSC-iNKT cells with SARS-CoV-2-infected cells (FIG. 2i). Overall, ^{Allo}HSC-iNKT cells demonstrated a potent capacity of direct killing of virus-infected cells and thereby may contribute to virus clearance.

### Elimination of Virus-Infection Promoted Inflammatory Monocytes by ^{Allo}HSC-iNKT Cells

Previous studies have indicated that iNKT cells could reduce accumulation of inflammatory monocytes in the lungs and decrease immunopathology under virus infection (28, 29). Therefore, we established another in vitro SARS-CoV-2 infection assay to study the elimination of virus-infection promoted inflammatory monocytes by ^{Allo}HSC-iNKT therapeutic cells via iNKT TCR/CD1d recognition (FIG. 3a). ^{Allo}HSC-iNKT cells effectively eliminated CD14⁺ inflammatory monocytes under SARS-CoV-2 infection condition, an effect that was reduced by blocking CD1d (FIGS. 3a-3c, 5c, and 5d). Meanwhile, T cells and B cells in the same cultures were not impacted, suggesting that ^{Allo}HSC-iNKT therapeutic cells will not compromise the T cell and B cell antiviral immunity important for combating COVID-19 (FIGS. 3b, 3c, 5c, and 5d) (50, 51). In agreement with an iNKT TCR/CD1d recognition-mediated mechanism, in the SARS-CoV-2 infection culture, inflammatory CD14⁺ monocytes expressed significantly higher levels of CD than that of T cells and B cells (FIGS. 3d and 3e) (28, 29). Therefore, ^{Allo}HSC-iNKT cells can potentially limit inflammation-mediated damage caused by virus infection by eliminating inflammatory monocytes (29).

### Safety Study of ^{Allo}HSC-iNKT Cells

Graft-versus-host (GvH) response is a main safety concern for “off-the-shelf” allogeneic cell therapies (52). Due to the expression of an invariant TCR targeting glycolipids presented by monomorphic MHC Class I-like CD1d molecules, iNKT cells do not react with mismatched HLA molecules and protein autoantigens, and are thus not expected to cause GvH response (33, 35). We used an established in vitro Mixed Lymphocyte Reaction (MLR) assay and an in vivo NSG mouse xenograft model to study the GvH response of ^{Allo}HSC-iNKT cells (FIGS. 4a and 4c). In contrast to conventional PBMC-Tc cells, ^{Allo}HSC-iNKT cells did not produce GvH responses against multiple mismatched-donor PBMCs, evidenced by their lack of IFN-γ secretion (FIG. 4b). In vivo, ^{Allo}HSC-iNKT cell-treated experimental mice did not have GvHD and sustained long-term survival, whereas PBMC-Tc cells-treated mice died of serious GvHD around two months post PBMC-Tc cell transfer (FIGS. 4c and 4d). In vitro and in vivo, ^{Allo}HSC-iNKT cells did not display GvHD risk.

### Immunogenicity Study of ^{Allo}HSC-iNKT Cells

For allogeneic cell products, immunogenicity is another concern due to potential allorejection by host T cells (53). Host conventional CD8 and CD4 αβ T cells target allogeneic cells through recognizing mismatched HLA-I and HLA-II molecules, respectively, and can greatly decrease the efficacy of therapeutic allogeneic cells (54, 55). In an in vitro MLR assay studying T cell-mediated host-versus-graft (HvG), ^{Allo}HSC-iNKT cells elicited significantly less IFN-γ secretion, a surrogate for HvG response, compared to PBMC-Tc cells (FIGS. 4e and 4f). Flow cytometry analysis showed that ^{Allo}HSC-iNKT cells expressed significantly reduced levels of HLA-I molecules than PBMC-Tc cells and nearly undetectable HLA-II molecules, indicating potential mechanisms for their resistance to T cell-mediated HvG responses (FIG. 4g). Because a virus infection-induced inflammatory microenvironment may upregulate the expression of HLA molecules on immune cells (e.g., via IFN-γ) (56), we also analyzed HLA expression on ^{Allo}HSC-iNKT cells in the presence of SARS-CoV-2 infection (FIG. 4h). As shown by flow cytometry analysis, under virus infection conditions ^{Allo}HSC-iNKT cells maintained low expressions of HLA-I and HLA-II molecules (FIGS. 4i and 4j). Cumulatively, these studies demonstrated the stable, low level expression of HLA-I and HLA-II molecules on ^{Allo}HSC-iNKT cells that may grant them resistance to host T cell-mediated rejection. The high safety and low immunogenicity features of ^{Allo}HSC-iNKT cells greatly support their application for “off-the-shelf” cell therapy.

### Discussion

The case and death tolls due to SARS-CoV-2 infection continue to rise as we enter what appears to be another wave of COVID-19 (1). The rapid, landmark development of highly effective vaccines (3-5) forms a crucial line of defense against COVID-19, but significant portions of society, for medical, accessibility, and other reasons, are unvaccinated (9). Additionally, breakthrough cases occur (7), emergent virus strains threaten vaccine efficacy (8, 57), and the duration of protection engendered by infection or vaccination is unknown (9). An off-the-shelf, variant-agnostic COVID-19 intervention is urgently needed to reduce patient mortality and protect vulnerable populations, and to provide a crucial window for the distribution of vaccines and potential subsequent doses (58).

Severe COVID-19 usually begins about one week after the onset of symptoms, and often manifests clinically as progressive respiratory failure that develops soon after dyspnea and hypoxemia (59, 60). These patients commonly suffer acute respiratory distress syndrome (ARDS), and can also experience lymphopenia, thromboembolic complications, disorders of the central or peripheral nervous system, acute cardiac, kidney, and liver injury, shock, and death (59, 60). The resulting organ failures correlate with signs of inflammation, including high fevers, heightened levels of proinflammatory cytokines and chemokines (i.e. IL-6, IL-8, MCP-1, CRP), and abnormal myeloid cell expansion and lung infiltration (61-63). Thousands of clinical COVID-19 trials testing antiviral compounds, immunomodulators, neutralizing agents, combination therapies, and other therapies have been initiated (64). To date, the FDA has solely approved remdesivir for severe COVID-19 treatment, although a survival benefit was not reported (18).

Cell-based immunotherapies have recently transformed the clinical landscape of blood malignancies (47, 65-67) and are an active area of research for antiviral treatments, including COVID-19 (16, 17). Invariant natural killer T (iNKT) are a rare, unique subpopulation of T cells that target lipid-based antigens presented by monomorphic MHC Class I-like CD1d molecules and have potent immunoregulatory functions (26-28). iNKT cell therapy has proven safe with signs of clinical activity in combatting cancer (68), and accumulating evidence suggests iNKT cells can ameliorate respiratory viral infection (28, 69, 70). In a model of mild influenza virus (IAV) infection, activation of iNKT cells reduced viral titers in the lungs of mice without affecting T cell immunity and was accompanied by a better disease course with improved weight loss profile (30). Using models of lethal, high pathogenicity influenza infection, Santo et. al. and Kok et. al. demonstrated that the absence of invariant NKT (iNKT) cells in mice during IAV infection resulted in the expansion of myeloid cells and correlated with increased viral titer, lung injury, and mortality. Activation or adoptive transfer of iNKT cells abolished the suppressive activity of myeloid cells, restored influenza-specific immune responses, reduced IAV titer, and increased survival rate, and the crosstalk between iNKT and myeloid cells was CD1d-dependent (29, 31). The results were extended to humans by demonstrating that the suppressive activity of myeloid cells present in the peripheral blood of individuals infected with influenza was substantially reduced by iNKT cell activation (31). In another preclinical mouse model, Paget et. al. showed that iNKT cells limit influenza pathology in a preclinical mouse model through the production of IL-22 (32). Importantly, a recent publication reporting on critically ill COVID-19 patients showed that the patients contained significantly reduced numbers of iNKT cells and the activation status of iNKT cells was predictive of disease severity, suggesting the involvement of iNKT cells in COVID-19 (25). Collectively, these studies indicate that iNKT cells play an important and beneficial role in battling acute respiratory virus infection, through mediating virus clearance and supporting effector responses while also limiting the degree of lung injury by regulating other immune responses and virus-mediated sequelae.

A critical bottleneck in the clinical application of iNKT cells is their scarcity, as iNKT cells make up ˜0.001-1% of peripheral blood cells. Two years ago, we reported the in vivo production of invariant natural killer T cells (iNKT) cells through TCR engineering of hematopoietic stem cells followed by bone marrow transfer. Advances in the Ex Vivo HSC-iNKT differentiation culture methods have allowed us to mature our platform into completely in vitro, CMC compliant systems that generate large quantities of pure, clonal iNKT cells (^{Allo}HSC-iNKT). Characterization of ^{Allo}HSC-iNKT cells revealed phenotypic and functional profiles comparable to endogenous peripheral blood iNKT cells, although ^{Allo}HSC-iNKT cells were predominated (97%) double negative (DN, CD4⁻CD8⁻) or CD8 single positive (SP). Mouse iNKT cells are CD4⁺ SP or DN, whereas human iNKT cells are CD4⁺ SP, CD8⁺ SP, or DN. In mice and human, CD4⁺ SP iNKT cells display a Th2 phenotype, favoring IL-4 production, whereas DN iNKT in mice and CD8⁺ SP and DN iNKT cells in humans are Th1-like and produce large quantities of IFN-γ. Of note, when assessed for CD4 expression, CD4⁻ and CD4⁺ iNKT cells were present in equal proportions in influenza-infected lungs but only CD4⁻ iNKT cells exhibited cytotoxicity towards inflammatory monocytes (29).

Using in vitro models, we have demonstrated the therapeutic potential of ^{Allo}HSC-iNKT cells against SARS-CoV-2 infection. Firstly, ^{Allo}HSC-iNKT cells lysed SARS-CoV-2-infected lung epithelial cells. Mechanistic analysis revealed NK-pathway mediated killing of SARS-CoV-2-infected cells, as the blocking of NKG2D or DNAM-1 receptors reduced target cell lysis (FIG. 2). In addition to the direct effect ^{Allo}HSC-iNKT cells can have on SARS-CoV-2 replication, ^{Allo}HSC-iNKT cells selectively eliminated virus-activated inflammatory monocytes. In the presence of SARS-CoV-2 infection, ^{Allo}HSC-iNKT cells lysed monocytes, without affecting T cell or B cell populations, in a CD1d-influenced manner (FIG. 3).

A major concern for allogeneic T cell-based therapies is GvHD (71), a potentially life-threatening disease in which donor T cells attack host tissue (72). By targeting non-polymorphic CD1d, iNKT cells avoid causing GvHD and have displayed an excellent safety profile in the clinic (68). Using mixed lymphocyte reactions and a preclinical mouse model, we did not observe GvH responses by ^{Allo}HSC-iNKT cells, whereas PBMC-derived T cells secreted IFN-γ in vitro and caused lethal GvHD in vivo (FIG. 4a-4d). It is also important that allogeneic cell therapies resist rejection by the host (i.e. HvG responses) to exert their therapeutic functions (71). ^{Allo}HSC-iNKT cells express remarkably low amounts of HLA-I and HLA-II molecules and maintained low expression of HLA-I and HLA-II molecules under SARS-CoV-2 infection (FIG. 4e-4j). Accordingly, in vitro MLRs showed that ^{Allo}HSC-iNKT cells are resistant to T cell-mediated allorejection.

Future studies testing iNKT cells in 3D human lung organoid infection models (73) and preclinical COVID-19 models will provide invaluable insights into the clinical application of ^{Allo}HSC-iNKT cells. Two potential in vivo models are a human lung xenograft NSG mouse infection model (74) and a hACE2 transgenic mouse infection model (75). The transgenic model will not support the direct study of human ^{Allo}HSC-iNKT cells due to xeno-incompatibility, and we plan to generate mouse HSC-engineered iNKT (mHSC-iNKT) cells as a therapeutic surrogate. Previously, we successfully generated mouse HSC-iNKT cells and showed that they closely resemble their native counterparts (38).

Our work underscores the potential for using iNKT cells to combat COVID-19, specifically TCR-engineered, HSC-derived iNKT cells. Using an Ex Vivo culture method, we generated thousands of ^{Allo}HSC-iNKT cell therapy doses from one cord blood donor. ^{Allo}HSC-iNKT cells can reduce SARS-CoV-2 pathogenicity through two distinct mechanisms: (1) direct killing of SARS-CoV-2 infected cells; (2) selective elimination of virus-activated inflammatory monocytes. Furthermore, ^{Allo}HSC-iNKT cells do not exhibit graft-versus-host responses and are resistant to immune cell allorejection, indicating ^{Allo}HSC-iNKT cells are a promising “off-the-shelf” anti-COVID-19 therapy.

### Methods

### Lentiviral Vectors and Transduction

The lentivector and lentivirus were generated as previously described (39). Lentiviral vectors used in this study were all constructed from a parental lentivector pMNDW. The Lenti/iNKT-sr39TK vector was constructed by inserting into pMNDW a synthetic tricistronic gene encoding human iNKT TCRa-F2A-TCRb-P2A-sr39TK; the Lenti/FG vector was constructed by inserting into pMNDW a synthetic bicistronic gene encoding Fluc-P2A-EGFP; the Lenti/ACE2 vector was constructed by inserting into pMNDW a synthetic gene encoding human ACE2. The synthetic gene fragments were obtained from GenScript and IDT. Lentiviruses were generated using 293T cells, following a standard calcium precipitation protocol and an ultracentrifugation concentration protocol or a tandem tangential flow filtration concentration protocol as previously described (38).

### SARS-CoV-2 Virus Generation

SARS-CoV-2, isolate USA-WA1/2020, was obtained from the Biodefense and Emerging Infections (BEI) Resources of the National Institute of Allergy and Infectious Diseases. All procedures involving SARS-CoV-2 infection were conducted within a Biosafety Level 3 facility at UCLA with appropriate institutional biosafety approvals. SARS-CoV-2 was passaged in African green monkey kidney epithelial cells (Vero E6; CRL-1586), which were maintained in D10 media comprised of Dulbecco's modified Eagle's medium (DMEM) supplemented with 10% fetal bovine serum (FBS; Omega Scientific) and 1% penicillin/streptomycin (P/S; Gibco). Viral stocks from the P6 passage were aliquoted and stored at −80° C. for this study. To assess viral titers, Vero E6 cells were infected and examined daily for cytopathic effect (CPE). TCID50 was calculated based on the method of Reed and Muench (76).

### Antibodies and Flow Cytometry

All flow cytometry stains were performed in PBS for 15 min at 4° C. The samples were stained with Fixable Viability Dye eFluor506 (e506) mixed with Mouse Fc Block (anti-mouse CD16/32) or Human Fc Receptor Blocking Solution (TrueStain FcX) prior to antibody staining. Antibody staining was performed at a dilution according to the manufacturer's instructions. Fluorochrome-conjugated antibodies specific for human CD45 (Clone H130), TCRαβ (Clone 126), CD4 (Clone OKT4), CD8 (Clone SK1), CD45RO (Clone UCHL1), CD161 (Clone HP-3G10), CD69 (Clone FN50), CD56 (Clone HCD56), CD62L (Clone DREG-56), CD14 (Clone HCD14), CD1d (Clone 51.1), NKG2D (Clone 1D11), DNAM-1 (Clone 11A8), IFN-γ (Clone B27), granzyme B (Clone QA16A02), perforin (Clone dG9), β2-microglobulin (B2M) (Clone 2M2), HLA-DR (Clone L243) were purchased from BioLegend; Fluorochrome-conjugated antibodies specific for human CD34 (Clone 581) and TCR Vα24-Jβ18 (Clone 6B11) were purchased from BD Biosciences. Unconjugated human ACE2 antibody was purchased from R&D Systems. Fluorochrome-conjugated Donkey anti-goat IgG was purchased from Abcam. Human Fc Receptor Blocking Solution (TrueStain FcX) was purchased from BioLegend, and Mouse Fc Block (anti-mouse CD16/32) was purchased from BD Biosciences. Fixable Viability Dye e506 were purchased from Affymetrix eBioscience. Intracellular cytokines were stained using a Cell Fixation/Permeabilization Kit (BD Biosciences). Flow cytometry were performed using a MACSQuant Analyzer 10 flow cytometer (Miltenyi Biotech) and data analyzed with FlowJo software version 9.

In Vitro Generation of Allogeneic HSC-Engineered iNKT (^{Allo}HSC-iNKT) Cells

Frozen-thawed human CD34⁺ HSCs were revived in HSC-culture medium composed of X-VIVO 15 Serum-free Hematopoietic Cell Medium supplemented with SCF (50 ng/ml), FLT3-L (50 ng/ml), TPO (50 ng/ml), and IL-3 (10 ng/ml) for 24 hours; the cells were then transduced with Lenti/iNKT-sr39TK viruses for another 24 hours following an established protocol (39). The transduced HSCs were then collected and cultured ex vivo to differentiate into iNKT cells,

via an Artificial Thymic Organoid (ATO) culture or a Feeder-Free culture. In the ATO culture, transduced HSCs were mixed with MS5-DLL4 feeder cells to form ATOs and cultured over ˜8 weeks following a previously established protocol (40, 41). In the Feeder-Free culture, transduced HSCs were cultured using a StemSpan T Cell Generation Kit (StemCell Technologies) over ˜5 weeks following the manufacturer's instructions. Differentiated ^{Allo}HSC-iNKT cells were then collected and expanded with αGC-loaded PBMCs for 1-2 weeks following a previously established protocol (39). The resulting ^{Allo}HSC-iNKT cell products were collected and cryopreserved for future use.
Generation of PBMC-Derived Conventional αβ T, and iNKT Cells

Healthy donor PBMCs were provided by the UCLA/CFAR Virology Core Laboratory and were used to generate the PBMC-Tc and PBMC-iNKT cells.

To generate PBMC-Tc cells, PBMCs were stimulated with CD3/CD28 T-activator beads (ThermoFisher Scientific) and cultured in C10 medium supplemented with human IL-2 (20 ng/mL) for 2-3 weeks, following the manufacturer's instructions.

To generate PBMC-iNKT cells, PBMCs were MACS-sorted via anti-iNKT microbeads (Miltenyi Biotech) labeling to enrich iNKT cells, which were then stimulated with donor-matched irradiated αGC-PBMCs at the ratio of 1:1, and cultured in C10 medium supplemented with human IL-7 (10 ng/ml) and IL-15 (10 ng/ml) for 2-3 weeks. If needed, the resulting PBMC-iNKT cells could be further purified using Fluorescence-Activated Cell Sorting (FACS) via human iNKT TCR antibody (Clone 6B11; BD Biosciences) staining.

### Cell Phenotype and Functional Study

Phenotype and functionality of multiple types of cells were analyzed, including ^{Allo}HSC-iNKT, PBMC-iNKT, and PBMC-Tc cells. Phenotype of these cells was studied using flow cytometry, by analyzing cell surface markers including co-receptors (CD4 and CD8), NK cell markers (CD161), memory T cell markers (CD45RO), and NK activating receptors (NKG2D and DNAM-1). Capacity of cells to produce cytokine (IFN-γ) and cytotoxic factors (Perforin and Granzyme B) was measured using Cell Fixation/Permeabilization Kit (BD Biosciences). PBMC-Tc and PBMC-iNKT cells were included as FACS analysis controls.

### SARS-CoV-2 Infection

SARS-CoV-2 infection was performed as previously described (77). For SARS-CoV-2 infection, viral inoculum (MOI of 0.1 and 1) was prepared using serum-free medium. Culture medium was removed and replaced with 250 μL of prepared inoculum in each well. For mock infection, serum-free medium (250 μL/well) was added. The inoculated plates were incubated at 37° C. with 5% CO2 for 1 hour. The inoculum was spread by gently tilting the plate sideways at every 15 minutes. At the end of incubation, the inoculum was replaced with fresh medium.

### In Vitro Killing Assay of SARS-CoV-2 Infected Target Cells

293T-FG, 293T-ACE2-FG, or Calu3-FG target cells (1×10³ cells per well) were seeded in Corning 96-well clear bottom black plates in D10 medium at day 0. At day 1, viral inoculum (MOI of 0.01) was prepared using D10 media. Media was gently removed without disrupting cells and replaced with 100 μl of prepared viral inoculum. ^{Allo}HSC-iNKT cells (1×10⁴ cells per well) were then added in the plates at day 2. At day 3 or day 4, live target cells were detected by using Luciferase Assay System (CAT #E1500, Promega) following its protocol. Medium was carefully removed from the wells, and 1× lysis reagent was added (20 μl per well) to lyse tumor cells and inactivate SARS-CoV-2 virus. Then the cell lysate was mixed with 50 μl of Luciferase Assay Reagent, and the luciferase activities were immediately analyzed using an Infinite M1000 microplate reader (Tecan). In the blocking assay, 10 μg per ml of LEAF′ purified anti-human NKG2D (Clone 1D11, Biolegend), anti-human DNAM-1 antibody (Clone 11A8, Biolegend), or LEAF™ purified mouse lgG2b κ isotype control antibody (Clone MG2B-57, Biolegend) was added to cell cultures when adding ^{Allo}HSC-iNKT cells at day 2.

### Enzyme-Linked Immunosorbent Cytokine Assays (ELISA)

The ELISA for detecting human cytokines were performed following a standard protocol from BD Biosciences. Supernatants from co-culture assays were collected, mixed with equal-volume 0.02% Triton™ X-100 reagent (Sigma-Aldrich), and assayed to quantify IFN-γ. Triton™ X-100 reagent was utilized for inactivating SARS-CoV-2 viruses. The capture and biotinylated pairs for detecting cytokines were purchased from BD Biosciences. The streptavidin-HRP conjugate was purchased from Invitrogen. Human cytokine standards were purchased from eBioscience. Tetramethylbenzidine (TMB) substrate was purchased from KPL. The samples were analyzed for absorbance at 450 nm using an Infinite M1000 microplate reader (Tecan).

### Immunofluorescence Imaging

293T-FG, 293T-ACE2-FG, or Calu3-FG target cells (2×10³ cells per well) were seeded in Chamber Slides in D10 medium at day 0. SARS-CoV-2 viral inoculum were added in the plates at day 1. ^{Allo}HSC-iNKT cells (2×10⁴ cells per well) were added at day 2. At day 4, supernatant was carefully removed. Cells were fixed in 4% paraformaldehyde (PFA) for 15 min, washed with PBS, followed by permeabilization and blocking in blocking buffer (PBS containing 0.1% Triton X-100 and 5% donkey serum) for 1 h at room temperature. Primary antibodies were diluted in blocking buffer and incubated with cells at 4° C. for overnight. The next day, cells were washed with PBS and incubated with secondary antibodies for 1 h at room temperature. Secondary antibodies were diluted in 1×PBS at 1:500 dilution. After incubation, cells were washed with PBS, incubated with DAPI (1:10,000) for 15 min, and mounted with Fluoromount-G reagent. The primary antibodies used include mouse anti CD3, 1:500 and mouse anti SARS-CoV-2 Spike, 1:200.

### In Vitro Mixed Lymphocyte Reaction (MLR) Assay: Studying Elimination of SARS-CoV-2 Infection Promoted Inflammatory Monocytes

293T-FG, 293T-ACE2-FG, or Calu3-FG target cells (1×10³ cells per well) were seeded in Corning 96-well clear bottom black plates in D10 medium at day 0. At day 1, viral inoculum (MOI of 0.01) was prepared using D10 media. Media was gently removed without disrupting cells and replaced with 100 μl of prepared viral inoculum. ^{Allo}HSC-iNKT cells (1×10⁴ cells per well) and donor-mismatched PBMCs were (1×10⁴ cells per well) were added in the plates at day 2. At day 3 or day 4, cells were analyzed by using flow cytometry. The culture supernatant was carefully removed from the wells, flow antibodies were added into the cells and incubated for 15 min on ice, and then the stained cells were fixed by 4% PFA for 1 hour. 4% PFA was also used here to inactivate SARS-CoV-2. Then flow cytometry was used to analyze the cell numbers and phenotypes. In the blocking assay, 10 μg per ml of LEAF™ purified anti-human CD1d (Clone 51.1, Biolegend), or LEAF™ purified mouse lgG2b κ isotype control antibody (Clone MG2B-57, Biolegend) was added to cell cultures at day 2.

### In Vitro Mixed Lymphocyte Reaction (MLR) Assay: Studying Graft-Versus-Host (GvH) Response

PBMCs of multiple healthy donors were irradiated at 2,500 rads and used as stimulators, to study the GvH response of ^{Allo}HSC-iNKT cells as responders. PBMC-Tc cells were included as a responder control. Stimulators (5×10⁵ cells/well) and responders (2×10⁴ cells/well) were co-cultured in 96-well round bottom plates in C10 medium for 4 days; the cell culture supernatants were then collected to measure IFN-γ production using ELISA.

### In Vitro MLR Assay: Studying Host-Versus-Graft (HvG) Response

PBMCs of multiple healthy donors were used as responders, to study the HvG response of ^{Allo}HSC-iNKT cells as stimulators (irradiated at 2,500 rads). PBMC-Tc cells were included as a stimulator control. Stimulators (5×10⁵ cells/well) and responders (2×10⁴ cells/well) were co-cultured in 96-well round bottom plates in C10 medium for 4 days; the cell culture supernatants were then collected to measure IFN-γ production using ELISA.

GvH Response of ^{Allo}HSC-iNKT cells in Human NSG Mouse Model

NSG mice (6-10 weeks of age) were pre-conditioned with 100 rads of total body irradiation, and then injected with 1×10^{7 Allo}HSC-iNKT cells or donor-matched PBMCs intravenously. Over time, mouse survival rates were recorded.

### Statistical Analysis

GraphPad Prism 6 (Graphpad Software) was used for statistical data analysis. Student's two-tailed t test was used for pairwise comparisons. Ordinary 1-way ANOVA followed by Tukey's multiple comparisons test was used for multiple comparisons. Log rank (Mantel-Cox) test adjusted for multiple comparisons was used for Meier survival curves analysis. Data are presented as the mean±SEM, unless otherwise indicated. In all figures and figure legends, “n” represents the number of samples or animals utilized in the indicated experiments. A P value of less than 0.05 was considered significant. ns, not significant; *P<0.05; **P<0.01; ***P<0.001; ****P<0.0001.

### CONCLUSION

This concludes the description of embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

### REFERENCES

1. Dong E, Du H, Gardner L. An interactive web-based dashboard to track COVID-19 in real time. Lancet. Infect. Dis. 2020. p. 533-4.
2. Carvalho J, Campos P, Carrito M, Moura C, Quinta-Gomes A, Tavares I, et al. The Relationship Between COVID-19 Confinement, Psychological Adjustment, and Sexual Functioning, in a Sample of Portuguese Men and Women. J Sex Med. Netherlands; 2021; 18:1191-7.
3. Polack F P, Thomas S J, Kitchin N, Absalon J, Gurtman A, Lockhart S, et al. Safety and Efficacy of the BNT162b2 mRNA Covid-19 Vaccine. N Engl J Med. 2020; 383:2603-15.
4. Baden L R, El Sahly H M, Essink B, Kotloff K, Frey S, Novak R, et al. Efficacy and Safety of the mRNA-1273 SARS-CoV-2 Vaccine. N Engl J Med. 2021; 384:403-16.
5. Sadoff J, Gray G, Vandebosch A, Cardenas V, Shukarev G, Grinsztejn B, et al. Safety and Efficacy of Single-Dose Ad26.COV2.S Vaccine against Covid-19. N Engl J Med. 2021; 384:2187-201.
6. Kim J H, Marks F, Clemens J D. Looking beyond COVID-19 vaccine phase 3 trials. Nat Med. United States; 2021; 27:205-11.
7. CDC. COVID-19 Vaccine Breakthrough Infections Reported to CDC—. MMWR Morb Mortal Wkly Rep. 2021; 70:792-3.
8. McCallum M, Bassi J, Marco A De, Chen A, Walls A C, Iulio J Di, et al. SARS-CoV-2 immune evasion by variant B.1.427/B.1.429. bioRxiv Prepr. Serv. Biol. 2021.
9. Forni G, Mantovani A, Forni G, Mantovani A, Moretta L, Rappuoli R, et al. COVID-19 vaccines: where we stand and challenges ahead. Cell Death Differ. 2021; 28:626-39.
10. Wang M, Cao R, Zhang L, Yang X, Liu J, Xu M, et al. Remdesivir and chloroquine effectively inhibit the recently emerged novel coronavirus (2019-nCoV) in vitro. Cell Res. 2020; 30:269-71.
11. Holshue M L, DeBolt C, Lindquist S, Lofy K H, Wiesman J, Bruce H, et al. First case of 2019 novel coronavirus in the United States. N Engl J Med. 2020; 382:929-36.
12. Ahn D G, Shin H J, Kim M H, Lee S, Kim H S, Myoung J, et al. Current status of epidemiology, diagnosis, therapeutics, and vaccines for novel coronavirus disease 2019 (COVID-19). J Microbiol Biotechnol. 2020; 30:313-24.
13. B. S, K. S, K. W, W. P. Perspectives on monoclonal antibody therapy as potential therapeutic intervention for Coronavirus disease-19 (COVID-19). Asian Pacific J allergy Immunol. 2020;
14. Zhang C, Wu Z, Li J W, Zhao H, Wang G Q. The cytokine release syndrome (CRS) of severe COVID-19 and Interleukin-6 receptor (IL-6R) antagonist Tocilizumab may be the key to reduce the mortality. Int J Antimicrob Agents. Elsevier B.V.; 2020; 55.
15. Tian X, Li C, Huang A, Xia S, Lu S, Shi Z, et al. Potent binding of 2019 novel coronavirus spike protein by a SARS coronavirus-specific human monoclonal antibody. Emerg Microbes Infect. 2020; 9:382-5.
16. Golchin A. Cell-Based Therapy for Severe COVID-19 Patients: Clinical Trials and Cost-Utility. Stem Cell Rev Reports. Stem Cell Reviews and Reports; 2021; 17:56-62.
17. Market M, Angka L, Martel A B, Bastin D, Olanubi O, Tennakoon G, et al. Flattening the COVID-19 Curve With Natural Killer Cell Based Immunotherapies. Front Immunol. 2020; 11:1-23.
18. Rubin D, Chan-Tack K, Farley J, Sherwat A. FDA Approval of Remdesivir—A Step in the Right Direction. N Engl J Med (Internet). Massachusetts Medical Society; 2020; 383:2598-600. Available from: https://doi.org/10.1056/NEJMp2032369
19. Neelapu S S, Tummala S, Kebriaei P, Wierda W, Gutierrez C, Locke F L, et al. Chimeric antigen receptor T-cell therapy—assessment and management of toxicities. Nat Rev Clin Oncol. 2018; 15:47-62.
20. Raje N, Berdeja J, Lin Y, Siegel D, Jagannath S, Madduri D, et al. Anti-BCMA CAR T-Cell Therapy bb2121 in Relapsed or Refractory Multiple Myeloma. N Engl J Med. 2019; 380:1726-37.
21. Kruger S, Ilmer M, Kobold S, Cadilha B L, Endres S, Ormanns S, et al. Advances in cancer immunotherapy 2019—latest trends. J Exp Clin Cancer Res. 2019; 38:268.
22. Einsele H. Immunotherapy for CMV infection. Cytotherapy. England; 2002; 4:435-6.
23. Heslop H E, Leen A M. T-cell therapy for viral infections. Hematol Am Soc Hematol Educ Progr. United States; 2013; 2013:342-7.
24. Papadopoulou A, Krance R A, Allen C E, Lee D, Rooney C M, Brenner M K, et al. Systemic inflammatory response syndrome after administration of unmodified T lymphocytes. Mol Ther. 2014; 22:1134-8.
25. Jouan Y, Guillon A, Gonzalez L, Perez Y, Boisseau C, Ehrmann S, et al. Phenotypical and functional alteration of unconventional T cells in severe COVID-19 patients. J Exp Med. 2020; 217.
26. Bendelac A, Savage P B, Teyton L. The Biology of NKT Cells. Annu Rev Immunol. 2007; 25:297-336.
27. Bae E A, Seo H, Kim I K, Jeon I, Kang C Y. Roles of NKT cells in cancer immunotherapy. Arch Pharm Res (Internet). Pharmaceutical Society of Korea; 2019; 42:543-8. Available from: https://doi.org/10.1007/s12272-019-01139-8
28. Juno J A, Keynan Y, Fowke K R. Invariant NKT Cells: Regulation and Function during Viral Infection. PLoS Pathog. 2012; 8.
29. Kok W L, Denney L, Benam K, Cole S, Clelland C, McMichael A J, et al. Pivotal Advance: Invariant NKT cells reduce accumulation of inflammatory monocytes in the lungs and decrease immune-pathology during severe influenza A virus infection. J Leukoc Biol. 2012; 91:357-68.
30. Ho L P, Denny L, Luhn K, Teoh D, Clelland C, McMichael A J. Activation of invariant NKT cells enhances the innate immune response and improves the disease course in influenza A virus infection. Eur J Immunol. 2008; 38:1913-22.
31. Santo C De, Salio M, Masri S H, Lee L Y, Dong T, Speak A O, et al. Jci0836264. 2008; 118:1-13.
32. Paget C, Ivanov S, Fontaine J, Renneson J, Blanc F, Pichavant M, et al. Interleukin-22 is produced by invariant natural killer T lymphocytes during influenza A virus infection: Potential role in protection against lung epithelial damages. J Biol Chem. 2012; 287:8816-29.
33. Haraguchi K, Takahashi T, Hiruma K, Kanda Y, Tanaka Y, Ogawa S, et al. Recovery of Vα24+ NKT cells after hematopoietic stem cell transplantation. Bone Marrow Transplant. 2004; 34:595-602.
34. Fujii S ichiro, Shimizu K, Okamoto Y, Kunii N, Nakayama T, Motohashi S, et al. NKT cells as an ideal anti-tumor immunotherapeutic. Front Immunol. 2013; 4:1-7.
35. de Lalla C, Rinaldi A, Montagna D, Azzimonti L, Bernardo M E, Sangalli L M, et al. Invariant NKT Cell Reconstitution in Pediatric Leukemia Patients Given HLA-Haploidentical Stem Cell Transplantation Defines Distinct CD4+ and CD4− Subset Dynamics and Correlates with Remission State. J Immunol. 2011; 186:4490-9.
36. Godfrey D I, Berzins S P. Control points in NKT-cell development. Nat Rev Immunol. 2007; 7:505-18.
37. Krijgsman D, Hokland M, Kuppen P J K. The role of natural killer T cells in cancer-A phenotypical and functional approach. Front Immunol. 2018; 9.
38. Smith D J, Liu S, Ji S, Li B, McLaughlin J, Cheng D, et al. Genetic engineering of hematopoietic stem cells to generate invariant natural killer T cells. Proc Natl Acad Sci USA. 2015; 112:1523-8.
39. Zhu Y, Smith D J, Zhou Y, Li Y R, Yu J, Lee D, et al. Development of Hematopoietic Stem Cell-Engineered Invariant Natural Killer T Cell Therapy for Cancer. Cell Stem Cell (Internet). Elsevier Inc.; 2019; 25:542-557.e9. Available from: https://doi.org/10.1016/j.stem.2019.08.004
40. Montel-Hagen A, Seet C S, Li S, Chick B, Zhu Y, Chang P, et al. Organoid-Induced Differentiation of Conventional T Cells from Human Pluripotent Stem Cells. Cell Stem Cell. 2019; 24:376-389.e8.
41. Seet C S, He C, Bethune M T, Li S, Chick B, Gschweng E H, et al. Generation of mature T cells from human hematopoietic stem and progenitor cells in artificial thymic organoids. Nat Methods. 2017; 14:521-30.
42. Shukla S, Langley M A, Singh J, Edgar J M, Mohtashami M, Zúñiga-Pflücker J C, et al. Progenitor T-cell differentiation from hematopoietic stem cells using Delta-like-4 and VCAM-1. Nat Methods. 2017; 14:531-8.
43. Iriguchi S, Yasui Y, Kawai Y, Arima S, Kunitomo M, Sato T, et al. A clinically applicable and scalable method to regenerate T-cells from iPSCs for off-the-shelf T-cell immunotherapy. Nat Commun. 2021; 12:430.
44. Huijskens M J A J, Walczak M, Koller N, Briedé J J, Senden-Gijsbers B L M G, Schnijderberg M C, et al. Technical advance: ascorbic acid induces development of double-positive T cells from human hematopoietic stem cells in the absence of stromal cells. J Leukoc Biol. United States; 2014; 96:1165-75.
45. Themeli M, Kloss C C, Ciriello G, Fedorov V D, Perna F, Gonen M, et al. Generation of tumor-targeted human T lymphocytes from induced pluripotent stem cells for cancer therapy. Nat Biotechnol. 2013; 31:928-33.
46. Aftab B T, Sasu B, Krishnamurthy J, Gschweng E, Alcazer V, Depil S. Toward “off-the-shelf” allogeneic CART cells. Adv Cell Gene Ther. 2020; 3:1-11.
47. Rezvani K, Rouce R, Liu E, Shpall E. Engineering Natural Killer Cells for Cancer Immunotherapy. Mol Ther (Internet). Elsevier Ltd.; 2017; 25:1769-81. Available from: http://dx.doi.org/10.1016/j.ymthe.2017.06.012
48. Sommer C, Boldajipour B, Kuo T C, Bentley T, Sutton J, Chen A, et al. Preclinical Evaluation of Allogeneic CART Cells Targeting BCMA for the Treatment of Multiple Myeloma. Mol Ther (Internet). Elsevier Ltd.; 2019; 27:1126-38. Available from: https://doi.org/10.1016/j.ymthe.2019.04.001
49. Lin Q, Zhao J, Song Y, Liu D. Recent updates on CAR T clinical trials for multiple myeloma. Mol Cancer. Molecular Cancer; 2019; 18:1-11.
50. Tay M Z, Poh C M, Rénia L, MacAry P A, Ng L F P. The trinity of COVID-19: immunity, inflammation and intervention. Nat Rev Immunol. 2020; 20:363-74.
51. Vabret N, Britton G J, Gruber C, Hegde S, Kim J, Kuksin M, et al. Immunology of COVID-19: Current State of the Science. Immunity. 2020; 52:910-41.
52. Labanieh L, Majzner R G, Mackall C L. Programming CAR-T cells to kill cancer. Nat Biomed Eng (Internet). Springer US; 2018; 2:377-91. Available from: http://dx.doi.org/10.1038/s41551-018-0235-9
53. Lanza R, Russell D W, Nagy A. Engineering universal cells that evade immune detection. Nat Rev Immunol (Internet). Springer US; 2019; 19:723-33. Available from: http://dx.doi.org/10.1038/s41577-019-0200-1
54. Ren J, Liu X, Fang C, Jiang S, June C H, Zhao Y. Multiplex genome editing to generate universal CAR T cells resistant to PD1 inhibition. Clin Cancer Res. 2017; 23:2255-66.
55. Steimle V, Siegrist C A, Mottet A, Lisowska-Grospierre B, Mach B. Regulation of MHC class II expression by interferon-γ mediated by the transactivator gene CIITA.

Science (80-). 1994; 265:106-9.
56. Castro F, Cardoso A P, Gonçalves R M, Serre K, Oliveira M J. Interferon-gamma at the crossroads of tumor immune surveillance or evasion. Front Immunol. 2018; 9:1-19.
57. Kupferschmidt K, Wadman M. Delta variant triggers new phase in the pandemic. Science (80-) (Internet). 2021; 372:1375-6. Available from: https://www.sciencemag.org/lookup/doi/10.1126/science.372.6549.1375
58. Mahase E. Covid-19: Booster dose will be needed in autumn to avoid winter surge, says government adviser. BMJ. 2021; 372:n664.
59. Berlin D A, Gulick R M, Martinez F J. Severe Covid-19. N Engl J Med. 2020; 383:2451-60.
60. McElvaney O J, McEvoy N L, McElvaney O F, Carroll T P, Murphy M P, Dunlea D M, et al. Characterization of the inflammatory response to severe COVID-19 Illness. Am J Respir Crit Care Med. 2020; 202:812-21.
61. Merad M, Sugie T, Engleman E G, Fong L. In vivo manipulation of dendritic cells to induce therapeutic immunity. Blood. 2002; 99:1676-82.
62. Schulte-Schrepping J, Reusch N, Paclik D, Baßler K, Schlickeiser S, Zhang B, et al. Severe COVID-19 Is Marked by a Dysregulated Myeloid Cell Compartment. Cell. 2020; 182:1419-1440.e23.
63. Li S, Jiang L, Li X, Lin F, Wang Y, Li B, et al. Clinical and pathological investigation of patients with severe COVID-19. JCI Insight. 2020; 5:1-13.
64. Bugin K, Woodcock J. Trends in COVID-19 therapeutic clinical trials. Nat Rev Drug Discov (Internet). Springer US; 2021; 20:254-5. Available from: http://dx.doi.org/10.1038/d41573-021-00037-3
65. Cohen A D, Garfall A L, Stadtmauer E A, Melenhorst J J, Lacey S F, Lancaster E, et al. B cell maturation antigen-specific CAR T cells are clinically active in multiple myeloma. J Clin Invest. 2019; 129:2210-21.
66. Mikkilineni L, Kochenderfer J N. Chimeric antigen receptor T-cell therapies for multiple myeloma. Blood. 2017; 130:2594-602.
67. Rosenberg S A, Restifo N P. Adoptive cell transfer as personalized immunotherapy for human cancer. Science (80-). 2015; 348:62-8.
68. King L A, Lameris R, de Gruijl T D, van der Vliet H J. CD1d-Invariant Natural Killer T Cell-Based Cancer Immunotherapy: α-Galactosylceramide and Beyond. Front Immunol. 2018; 9.
69. Van Dommelen S L H, Degli-Esposti M A. NKT cells and viral immunity. Immunol Cell Biol. 2004; 82:332-41.
70. Newton A H, Cardani A, Braciale T J. The host immune response in respiratory virus infection: balancing virus clearance and immunopathology. Semin Immunopathol (Internet). Seminars in Immunopathology; 2016; 38:471-82. Available from: http://dx.doi.org/10.1007/s00281-016-0558-0
71. Depil S, Duchateau P, Grupp S A, Mufti G, Poirot L. ‘Off-the-shelf’ allogeneic CAR T cells: development and challenges. Nat Rev Drug Discov (Internet). Springer US; 2020; 19:185-99. Available from: http://dx.doi.org/10.1038/s41573-019-0051-2
72. Ferrara J L, Levine J E, Reddy P, Holler E. Graft-versus-host disease. Lancet (Internet). Elsevier Ltd; 2009; 373:1550-61. Available from: http://dx.doi.org/10.1016/S0140-6736(09)60237-3
73. van der Vaart J, Lamers M M, Haagmans B L, Clevers H. Advancing lung organoids for COVID-19 research. Dis Model Mech (Internet). 2021; 14:1-6. Available from: http://www.ncbi.nlm.nih.gov/pubmed/34219165%0Ahttp://www.pubmedcentral.nih.gov/articlerender.fcgi?artid=PMC 8272930
74. Valbuena G, Halliday H, Borisevich V, Goez Y, Rockx B. A Human Lung Xenograft Mouse Model of Nipah Virus Infection. PLoS Pathog. 2014; 10.
75. Bao L, Deng W, Huang B, Gao H, Liu J, Ren L, et al. The pathogenicity of SARS-CoV-2 in hACE2 transgenic mice. Nature. 2020; 583:830-3.
76. REED LJ, MUENCH H. A SIMPLE METHOD OF ESTIMATING FIFTY PERCENT ENDPOINTS12. Am J Epidemiol (Internet). 1938; 27:493-7. Available from: https://doi.org/10.1093/oxfordjournals.aje.a118408
77. Sharma A, Garcia G, Wang Y, Plummer J T, Morizono K, Arumugaswami V, et al. Human iPSC-Derived Cardiomyocytes Are Susceptible to SARS-CoV-2 Infection. Cell Reports Med (Internet). Elsevier Company.; 2020; 1:100052. Available from: https://doi.org/10.1016/j.xcrm.2020.100052

All publications mentioned herein (e.g. those listed numerically above and International Patent Application No PCT/US2020/037486) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A method of inhibiting replication of severe acute respiratory syndrome coronavirus 2 comprising:
combining a plurality of purified invariant natural killer T (iNKT) cells with cells infected with the severe acute respiratory syndrome coronavirus 2 such that the invariant natural killer T (iNKT) cells selectively kill the cells infected with the severe acute respiratory syndrome coronavirus 2, thereby inhibiting replication of severe acute respiratory syndrome coronavirus 2; wherein:
the plurality of purified invariant natural killer T (iNKT) cells comprise allogeneic CD34⁺ hematopoietic stem cell derived engineered invariant natural killer T (iNKT) cells.

2. The method of claim 1, wherein the engineered iNKT cells are obtained by transducing one or more exogenous nucleic acids into CD34⁺ stem or progenitor cells such that the engineered iNKT cells are made.

4. The method of claim 2, wherein:
the one or more exogenous nucleic acids transduced into the stem or progenitor cells comprise a T cell receptor alpha chain gene and/or a T cell receptor alpha chain gene; and
the engineered iNKT cells comprise clonal populations of cells comprising the T cell receptor alpha chain gene and/or the T cell receptor beta chain gene.

5. The method of claim 2, wherein the plurality of purified invariant natural killer T (iNKT) cells comprise engineered invariant natural killer T (iNKT) cells further comprise a gene expression profile characterized as:
HLA-I-negative;
HLA-II-negative;
HLA-E-positive; and
expressing a suicide gene.

6. The method of claim 1, wherein the plurality of purified invariant natural killer T (iNKT) cells comprises at least 1×10⁶ invariant natural killer T (iNKT) cells.

7. The method of claim 4, wherein the engineered iNKT cell comprises one or more exogenous nucleic acids encoding at least one functional T-cell receptor (TCR), wherein the TCR recognizes one or more SARS-CoV-2 antigens.

8. The method of claim 1, wherein the method comprises using a plurality of purified invariant natural killer T (iNKT) cells that are thawed following cryopreservation.

9. The method of claim 1, wherein the cells infected with the severe acute respiratory syndrome coronavirus 2 are human monocyte cells.

10. The method of claim 1, wherein the plurality of purified invariant natural killer T (iNKT) cells are combined in vivo with cells infected with the severe acute respiratory syndrome coronavirus 2 so as to treat an individual suffering from coronavirus disease 19.

11. A composition of matter comprising:
a plurality of purified invariant natural killer T (iNKT) cells; and
cells infected with the severe acute respiratory syndrome coronavirus 2; wherein:
the plurality of purified invariant natural killer T (iNKT) cells comprise allogeneic CD34⁺ hematopoietic stem cell derived engineered invariant natural killer T (iNKT) cells.

12. The composition of claim 11, wherein the engineered iNKT cells comprise one or more exogenous nucleic acids transduced therein.

13. The composition of claim 12, wherein:
the one or more exogenous nucleic acids comprise a T cell receptor alpha chain gene and/or a T cell receptor alpha chain gene; and
the engineered iNKT cells comprise clonal populations of cells comprising the T cell receptor alpha chain gene and/or the T cell receptor beta chain gene.

14. The composition of claim 12, wherein the plurality of purified invariant natural killer T (iNKT) cells comprise engineered invariant natural killer T (iNKT) cells further comprise a gene expression profile characterized as:
HLA-I-negative;
HLA-II-negative;
HLA-E-positive; and
expressing a suicide gene.

15. The composition of claim 11, wherein the plurality of purified invariant natural killer T (iNKT) cells comprises at least 1×10⁸ invariant natural killer T (iNKT) cells.

16. The composition of claim 12, wherein the engineered iNKT cell comprises one or more exogenous nucleic acids encoding at least one functional T-cell receptor (TCR), wherein the TCR recognizes one or more SARS-CoV-2 antigens.

18. The composition of claim 11, wherein the cells infected with the severe acute respiratory syndrome coronavirus 2 are human monocyte cells.

19. The composition of claim 11, wherein the plurality of purified invariant natural killer T cells express at least one of an invariant TCR α chain or a semi-variant TCR β chain.

20. The composition of claim 11, further comprising a pharmaceutically acceptable excipient selected from at least one of a preservative, a tonicity adjusting agent, a detergent, a hydrogel, a viscosity adjusting agent, or a pH adjusting agent.
